(19) United States
(12) Patent Application Publication
(10) Pub. No.: US 2025/0381005 A1
(43) Pub. Date: Dec. 18, 2025

(54) CABINET FOR ENDOSCOPE REPROCESSING

(71) Applicant: Boston Scientific Scimed, Inc., Maple Grove, MN (US)

(72) Inventors: Daniela Cespedes Valenzuela, Bogotá (CO); Santiago Grajales Londono, Bogotá (CO); Juan Pablo Herrera, Bogotá (CO); Santiago Sanchez Renteria, Bogotá D.C (CO); Carolina Villarreal, Hopedale, MA (US); Andrés Felipe Manrique Moreno, Bogotá (CO)

(73) Assignee: Boston Scientific Scimed, Inc., Maple Grove, MN (US)

(21) Appl. No.: 19/234,779

(22) Filed: Jun. 11, 2025

Related U.S. Application Data
(60) Provisional application No. 63/659,199, filed on Jun. 12, 2024.

(51) Int. Cl. A61B 50/10; A61L 2/02; A61L 2/10; A61L 2/26

(52) U.S. Cl. CPC A61B 50/10; A61L 2/022; A61L 2/10; A61L 2/26; A61B 2050/105; A61L 2202/11; A61L 2202/121; A61L 2202/122; A61L 2202/16; A61L 2202/24

(57) ABSTRACT

Devices, systems, and methods for reprocessing endoscopic devices include a cabinet for drying and holding disinfected endoscopes after use. A protective layer surrounds a frame that holds the endoscopes. The frame can be assembled by hand. The protective layer includes a filtered air inlet and filtered air outlet. UV lights aid the disinfection and drying of the endoscopes.

### CROSS-REFERENCE TO RELATED APPLICATIONS

This application claims the benefit of U.S. Provisional Patent Application Ser. No. 63/659,199 filed on Jun. 12, 2024, the disclosure of which is incorporated herein by reference.

### FIELD

This disclosure relates generally to cleaning devices and methods, and particularly for cleaning devices for an endoscope.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages.

Because of the high costs associated with manufacturing these components, many are designed to be reusable. However, the reuse of endoscopic medical devices is in tension with the need for a sterile medical environment. There is therefore a need for efficient and effective procedures to thoroughly clean and sanitize endoscopic devices prior to reuse.

### SUMMARY

This disclosure provides design, material, manufacturing method, and use alternatives for medical devices and medical systems. In a first example, a cabinet for endoscope reprocessing includes a frame supporting a plurality of endoscope holders, the frame defining an interior volume of the cabinet; and a removable protective layer surrounding the frame, the removable protective layer having a filtered air inlet and a filtered air outlet.

Alternatively or additionally to any of the examples above, the cabinet can further include at least one ultraviolet lamp radiating ultraviolet light into the interior volume of the cabinet.

Alternatively or additionally to any of the examples above, the cabinet can further include a removable drip tray positioned below the plurality of endoscope holders. The removable drip tray can have a plurality of anti-splash contours.

Alternatively or additionally to any of the examples above, the removable drip tray can sit on the removable protective layer and can be not directly attached to the frame.

Alternatively or additionally to any of the examples above, the frame can include modular components configured for repeated assembly and disassembly by hand. The modular components can be PVC crosspieces and joints.

Alternatively or additionally to any of the examples above, the removable protective layer can include flexible, air-impermeable sheets of material isolating the interior volume of the cabinet from air surrounding the cabinet.

Alternatively or additionally to any of the examples above, the filtered air inlet and/or the filtered air outlet can have a HEPA filter.

Alternatively or additionally to any of the examples above, each of the plurality of endoscope holders can include a clip sized and shaped to securely hold an endoscope. The clip can be sized and shaped to vertically suspend an endoscopic handle, allowing tubing to hang downward from the handle.

In another example, a method for reprocessing a plurality of endoscopes includes the steps of disinfecting each of the plurality of endoscopes and placing each of the disinfected endoscopes into a cabinet according to any of the examples above.

Alternatively or additionally to any of the examples above, the method can further include the steps of removing one endoscope of the plurality of disinfected endoscopes from the cabinet and using the endoscope removed from the cabinet in an endoscopic procedure.

Alternatively or additionally to any of the examples above, the disinfecting step can include High-Level Disinfection.

Alternatively or additionally to any of the examples above, the cabinet can further include at least one ultraviolet lamp radiating ultraviolet light into the interior volume of the cabinet.

Alternatively or additionally to any of the examples above, the cabinet can further include a removable drip tray positioned below the plurality of endoscope holders.

Alternatively or additionally to any of the examples above, the cabinet frame can further include modular components configured for repeated assembly and disassembly by hand.

Alternatively or additionally to any of the examples above, the method can further include, prior to placing the plurality of endoscopes into the cabinet, assembling the frame of the cabinet by hand.

Alternatively or additionally to any of the examples above, the method can further include the steps of: removing the plurality of endoscopes from the cabinet; disassembling the cabinet, including disassembling the frame of the cabinet by hand; transporting the disassembled cabinet to different location; and reassembling the cabinet in the different location.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3 is a flowchart illustrating an exemplary medical device cleaning process;

FIG. 4 depicts a perspective view of an illustrative cabinet;

FIG. 5 depicts a perspective view of an illustrative protective cover;

FIGS. 6A-6C depict perspective views of parts of an illustrative cabinet;

FIG. 7 depicts an illustrative drip tray; and

FIG. 8 depicts an illustrative endoscope holder.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

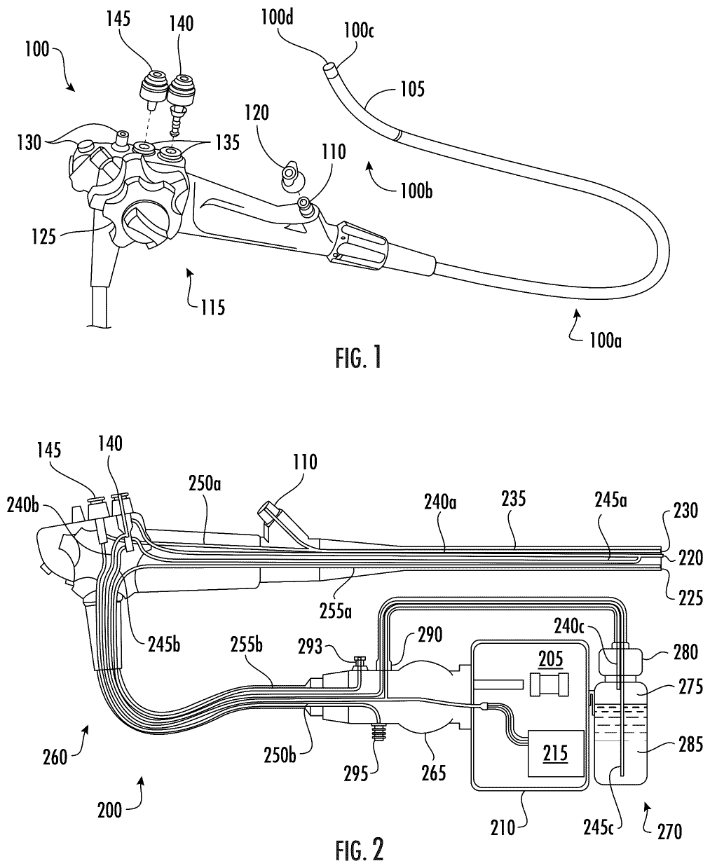

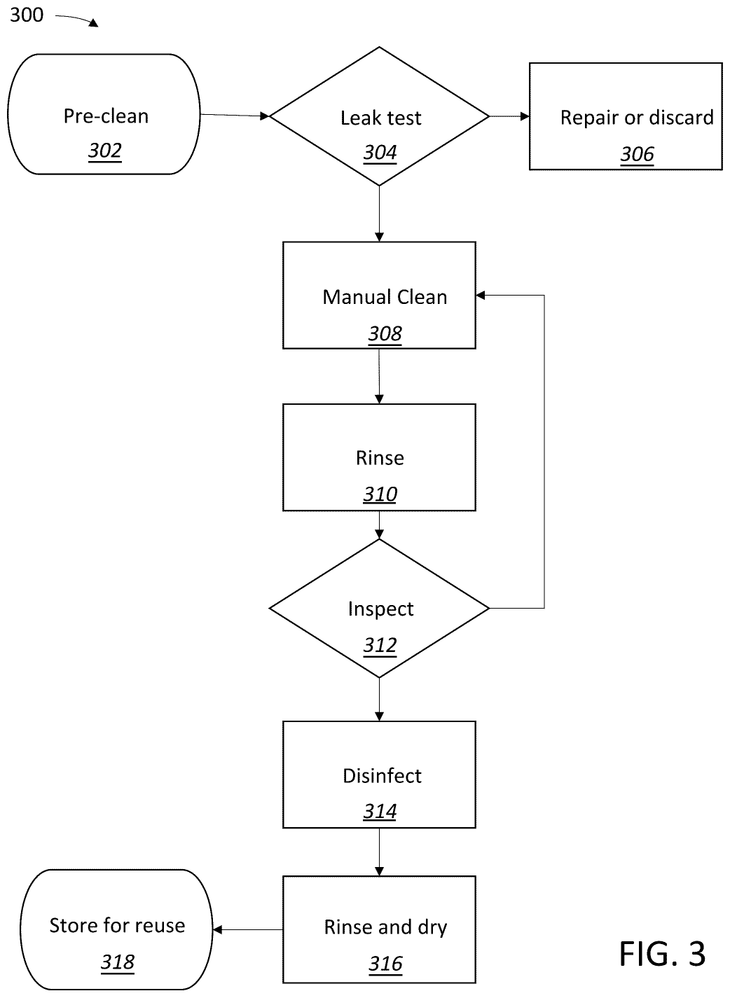

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serves as a component of an air/water feed circuit by housing a pressurizing pump 215, such as an air feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

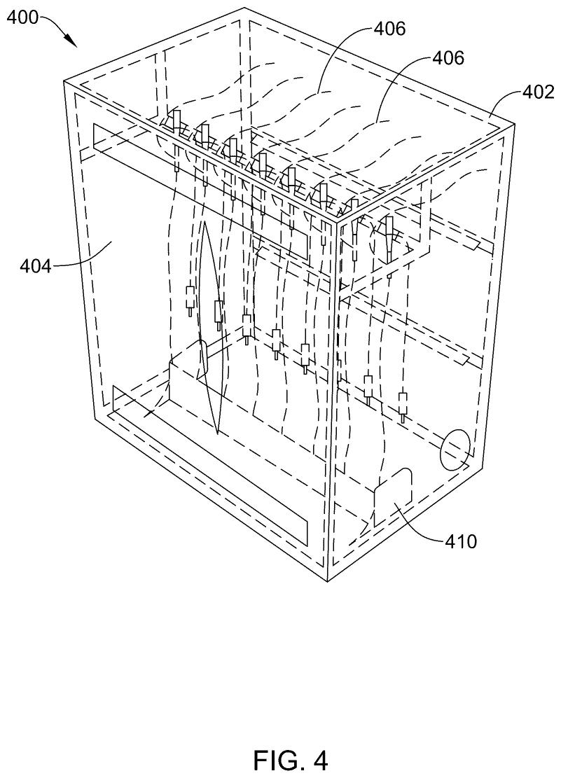

After the use of the above system 200 in an endoscopic procedure, some of the components may be reprocessed for subsequent reuse. An example of such a reprocessing method 300 is shown in FIG. 3.

In step 302, components of the system are subject to initial cleaning. This pre-cleaning step may be carried immediately after the endoscopic procedure or at some later time. Pre-cleaning may involve an initial rinse and/or immersion to rid components of any visible external fluid or detritus. In some implementations, pre-cleaning may be performed at a cleaner's discretion based on informal or casual inspection of the components to be cleaned.

Following pre-cleaning, in step 304, leak testing may be performed on one or more of the components, particularly the tubing. This may involve running a sterile liquid, such as water or saline solution, through the supply and/or working channels to ensure that none escapes. Should any component be found to have a leak, it may be set aside for disposal or repair (step 306), and only components that pass the leak testing complete the remaining steps of the reprocessing as described.

A manual cleaning is performed at step 308. Here, brushes and other tools are used to remove any debris. It is during this manual cleaning step that, in some implementations, a cleaning brush of the sort described herein may be used. The handle 115 and any of the tubing described herein may be cleaned. Particularly the working channel 235 and any other tubing that exposed to biological material such as patient fluids, samples, or waste may be included in the manual cleaning. For each channel to be cleaned, cleaning tools such as the cleaning brush are inserted and pushed entirely through the channel to capture and remove the debris in that channel.

One or more rinsing cycles follow the manual cleaning at step 310, which may involve any appropriate cleaning liquid such as distilled water, saline, detergent solution, or the like. A visual inspection is then performed (step 312) to confirm the efficacy of the cleaning and rinsing steps; a failed inspection may then require more manual cleaning (returning to step 308).

Should the inspection confirm that the component or components are satisfactorily free of debris, the devices may then be disinfected at step 314. The disinfecting process may involve the exposure of components to radiation, temperature, liquids, gases, or any combination of these, and may be carried out over any time period sufficient to thoroughly clean the device to the standards necessary for medical reuse. In some implementations, the disinfection process may meet regulatory and industry standards for High-Level Disinfection.

Following the disinfection process, the components are dried (step 316) and stored for reuse (step 318). To maintain the components' status as disinfected and available for surgical use, the drying and storage must occur under sterile conditions. The components should be maintained in a sterile environment, and any air used to dry the component should likewise meet any appropriate standards of source and filtration.

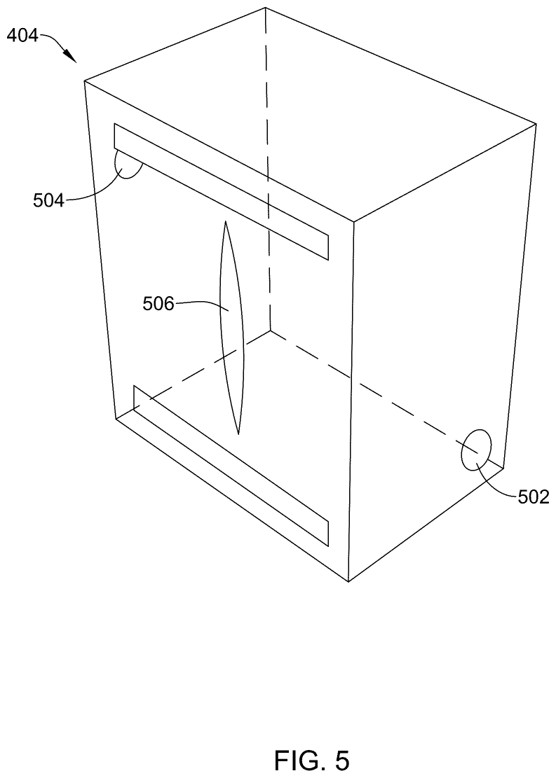

A cabinet for use in the drying and storage steps is shown in FIG. 4. A drying cabinet 400 is formed with a frame 402, which may be formed with modular components such as PVC piping and fasteners. A protective layer 404 is placed around the frame 402. The protective layer 404 can be any barrier capable of sealing the interior against the environment. The protective layer 404 may be, for example, made of flexible plastic sheeting. Endoscopes are hung along an interior cross-piece of the frame 402, each connected to air line 406 A drip tray 410 sits below the endoscopes to collect fluids during drying.

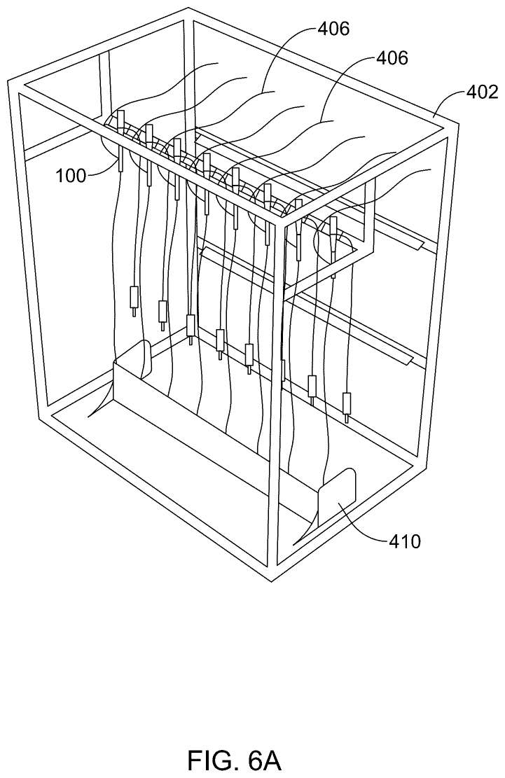

As shown in FIG. 5, the protective layer 404 includes an air inlet 502 and an air outlet 504. These air openings 502, 504 each include air filters which meet HEPA or other relevant standards for filtration. The filters at the inlet 502 and outlet 504 allow the interior of the cabinet 400 to be considered a sterilized environment during drying and storage of the endoscopic components. The inlet 502 and outlet 504 can be sized for the accommodation of existing infrastructure. For example, where a negative-pressure suction source is provided as in many medical environments, the suction source can be attached to the filter at the air outlet 504, drawing air from the room through the filter at the air inlet 502. Alternatively, where a positive-pressure air source is provided, the air source can be attached to the filter at the air inlet 502, expelling air through the filter at the air outlet 504.

The front of the protective layer 404 has an opening 506 for adding and removing components from the cabinet 400. The opening 506 may use any appropriate fastening or mechanism to maintain the sterile environment when not being used. In some implementations, the material of the protective layer 404 may provide sufficient elasticity and resilience to allow a user access through the opening 506 while reverting to a closed state when not in use. Variations of the opening for the protective layer 404 will be recognized by one of ordinary skill, including the use of one or more overlapping plastic sheets at or near the opening.

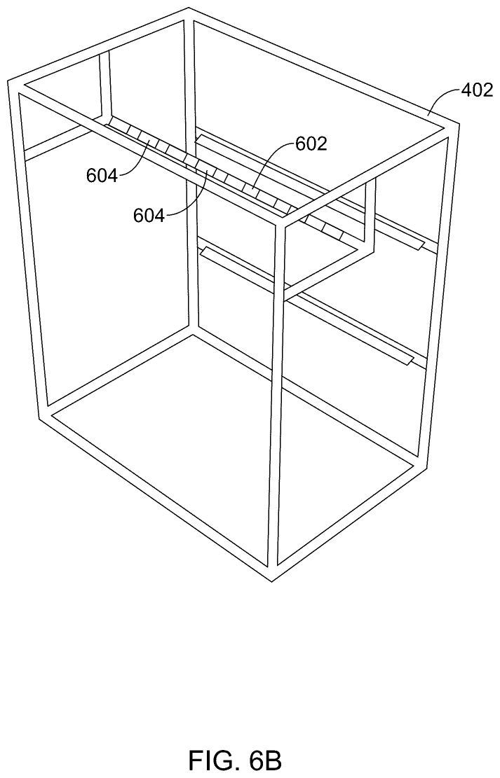

FIGS. 6A, 6B, and 6C depict the frame 402 or a portion of the frame 402 on which multiple endoscopes 100 may be hung for drying and storage. A center crosspiece 602 includes endoscope holders 604 spaced along the length of the frame 402 at sufficient distance to allow each of the hung devices to dry. The separately removable drip tray 410 is positioned below the holders 604. The drip tray 410 is removed and cleaned regularly to avoid cross-contamination.

Each endoscope 100 is connected to an air line 406 to facilitate drying of the endoscope channels. Each line 406 may connect to the endoscope handle at a fluid input location, connecting in place of an air, water, or suction supply line while the endoscope 100 is drying. The air lines 406 may connect to an exterior air supply, such as a hospital room air supply. One or more small openings (not shown) in the protective layer 404 may accommodate the air lines 406.

As shown, the entirety of the frame 402 is made of joints and crosspieces of uniform dimension and relatively low cost. The entire cabinet 400 can be transported unassembled as a much smaller package, then quickly and easily assembled on-site with minimal tools.

UV lamps 606 are attached to rear crosspieces of the frame in FIGS. 6A-6C (labeled in FIG. 6C). These UV lamps may be battery-operated or may connect to an external power source. As shown, the UV lamps 606 may be removably attached by means of clips or other fasteners for easy assembly and disassembly if needed. The lamps 606 may inhibit microbial growth within the cabinet 400.

While a set of four UV lamps is illustrated, one of ordinary skill will recognize that other radiation sources may be used within the cabinet 400. For example, infrared lamps may be used to facilitate rapid drying of the endoscopes. A combination of radiation sources may also be used, and the power and frequency of radiation may be varied over time as endoscopes are added and removed from the cabinet.

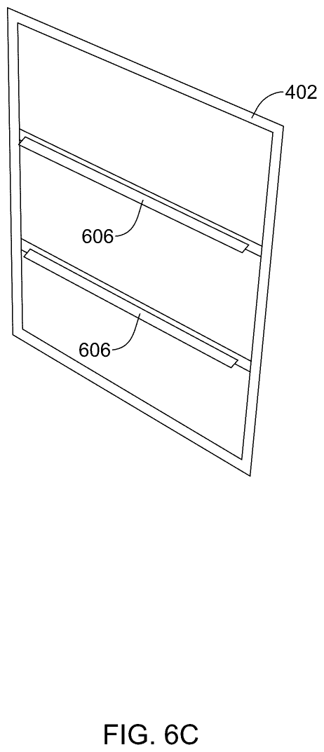

The drip tray 410 may have contours 702 as shown in FIG. 7. The contours 702 are selected to minimize splash, assuring that most moisture remains in the tray rather than the floor of the cabinet. Legs 704 brace the drip tray 410, assuring stability. The drip tray 410 may be made of any material appropriate for receiving and storing water and may include antimicrobial properties. In some implementations, the drip tray 410 may have surfaces suitable for reflecting radiation within the cabinet 400, directing more of the radiation from the lamps 606 or other radiation sources to the drying components.

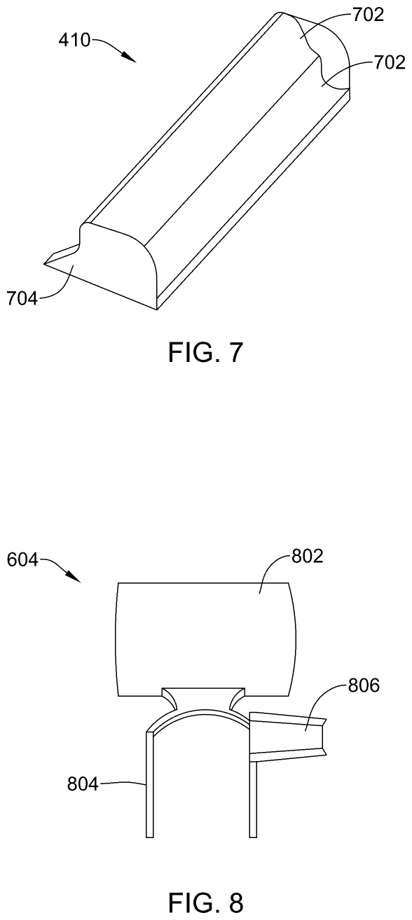

As shown in FIG. 8, a holder 604 is sized and shaped to easily receive and hold an endoscope. A cylindrical portion 802 interfaces with the crosspieces of the frame 402 for solid assembly. A clip 804 projects horizontally from the cylindrical portion 802 securely holds the body of the endoscopic handle 115 and includes a groove 806 also conforming to the outer casing. Where multiple shapes or configurations of endoscopes are used, one or more different configurations of holder may be substituted within the frame.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A cabinet for endoscope reprocessing, comprising:
a frame supporting a plurality of endoscope holders, the frame defining an interior volume of the cabinet; and
a removable protective layer surrounding the frame, the removable protective layer having a filtered air inlet and a filtered air outlet.

2. The cabinet of claim 1, further comprising at least one ultraviolet lamp radiating ultraviolet light into the interior volume of the cabinet.

3. The cabinet of claim 1, further comprising a removable drip tray positioned below the plurality of endoscope holders.

4. The cabinet of claim 3, wherein the removable drip tray comprises a plurality of anti-splash contours.

5. The cabinet of claim 3, wherein the removable drip tray sits on the removable protective layer and is not directly attached to the frame.

6. The cabinet of claim 1, wherein the frame comprises modular components configured for repeated assembly and disassembly by hand.

7. The cabinet of claim 6, wherein the modular components are PVC crosspieces and joints.

8. The cabinet of claim 1, wherein the removable protective layer comprises flexible, air-impermeable sheets of material isolating the interior volume of the cabinet from air surrounding the cabinet.

9. The cabinet of claim 1, the filtered air inlet having a HEPA filter.

10. The cabinet of claim 1, the filtered air outlet having a HEPA filter.

11. The cabinet of claim 1, wherein each of the plurality of endoscope holders comprises a clip sized and shaped to securely hold an endoscope.

12. The cabinet of claim 11, wherein the clip is sized and shaped to vertically suspend an endoscopic handle, allowing tubing to hang downward from the handle.

13. A method for reprocessing a plurality of endoscopes, comprising:
disinfecting each of the plurality of endoscopes; and
placing each of the disinfected endoscopes into a cabinet comprising:
a frame supporting a plurality of endoscope holders, the frame defining an interior volume of the cabinet; and
a removable protective layer surrounding the frame, the removable protective layer having a filtered air inlet and a filtered air outlet.

14. The method of claim 13, further comprising:
removing one endoscope of the plurality of disinfected endoscopes from the cabinet; and
using the endoscope removed from the cabinet in an endoscopic procedure.

15. The method of claim 13, wherein the disinfecting step comprises High-Level Disinfection.

16. The method of claim 13, the cabinet further comprising at least one ultraviolet lamp radiating ultraviolet light into the interior volume of the cabinet.

17. The method of claim 13, the cabinet further comprising a removable drip tray positioned below the plurality of endoscope holders.

18. The method of claim 13, wherein the cabinet frame comprises modular components configured for repeated assembly and disassembly by hand.

19. The method of claim 18, further comprising:
prior to placing the plurality of endoscopes into the cabinet, assembling the frame of the cabinet by hand.

20. The method of claim 19, further comprising:
removing the plurality of endoscopes from the cabinet;
disassembling the cabinet, including disassembling the frame of the cabinet by hand;
transporting the disassembled cabinet to different location; and
reassembling the cabinet in the different location.
